(19) United States
(12) Patent Application Publication
(10) Pub. No.: US 2008/0119762 A1
(43) Pub. Date: May 22, 2008

(54) GUIDE WIRE

(76) Inventors: Tadasu TATEISHI, Hadano-shi (JP); Yuuki Yoshifusa, Fujinomiya-shi (JP); Katsuhiro Shirakawa, Fujinomiya-shi (JP); Youki Aimi, Fujinomiya-shi (JP)

Correspondence Address: BUCHANAN, INGERSOLL & ROONEY PC, POST OFFICE BOX 1404, ALEXANDRIA, VA, 22313-1404, US

(21) Appl. No.: 11/859,370

(22) Filed: Sep. 21, 2007

Related U.S. Application Data
(60) Provisional application No. 60/877,652, filed on Dec. 29, 2006.

(30) Foreign Application Priority Data
Nov. 16, 2006 (JP) 2006-310006
Feb. 28, 2007 (JP) 2007-48942

(51) Int. Cl. A61M 25/09; B21D 31/06

(52) U.S. Cl. 600/585; 72/364

(57) ABSTRACT

A guide wire includes a wire body, a wire distal end section at the distal side of the wire body and integrally formed in one piece with the wire body, a flat plate-shaped section provided at a distal end section of the guide wire and at the wire distal end section, a coating layer formed on a surface of the wire body, and a coil mounted at the wire distal end section. A surface part of the flat plate-shaped section preferably has a residual stress higher than that an internal part of the flat plate-shaped section.

This application claims priority under 35 U.S.C. § 119(e) with respect to U.S. provisional Application No. 60/877,652 filed on Dec. 29, 2006, and is also based on and claims priority under 35 U.S.C. § 119(a) with respect to Japanese Application No. 2007-48942 filed on Feb. 28, 2007, the entire content of both of which is incorporated herein by reference.

### TECHNICAL FIELD

The disclosed subject matter generally pertains to a guide wire, and more specifically to a guide wire used in introducing a catheter into a body cavity such as a blood vessel and a bile duct.

### BACKGROUND DISCUSSION

Conventionally, the introduction of a catheter into a blood vessel is carried out for test or therapy of a cardiac disease or the like. In introducing a catheter to a target site in a body, a guide wire is inserted in the catheter, and a distal end (tip) section of the guide wire is moved forward prior to the catheter. After the distal end section of the guide wire has reached the target site, the catheter is guided to the target site over the guide wire.

Considering the procedure carried out during PCI (Percutaneous Coronary Intervention), the distal end section of a guide wire is moved forward while selecting a branch of coronary arteries under fluoroscopic observation until the distal end section reaches a stenosis portion, which is the target site, and then the distal end section is passed through the stenosis portion. Thereafter, a dilation catheter provided with a balloon at the distal end thereof is guided along the guide wire to locate the balloon of the dilation catheter at the stenosis portion. Then, the balloon is dilated to dilate the stenosis portion so as to secure a quantity of bloodstream, thereby treating stenocardia or the like.

In order to insert a guide wire from a femoral artery and move it forward through an aorta, an aortic arch and a coronary artery orifice into the coronary artery, it is desirable that the guide wire possesses sufficient flexibility for following the curvature of the blood vessels and so that the pushing force is transmitted from the operator's hand (the proximal side) to the distal end part of the guide wire.

For the purpose of moving the guide wire forward into a desired branch at a branching part of the coronary artery, a distal end part of the guide wire is manually shaped into a shape generally conforming to the shape of the branching part. Such an operation is called “reshaping”. When inserting a guide wire into the coronary artery on the peripheral side, for example, it is quite difficult, is not impossible, to select the desired branch while using the angle-type or J-type tip shape of the guide wire preformed according to known guide wires. Therefore, in many cases, the guide wire tip must be reshaped into a desired shape before inserting the guide wire again. When the shape of the guide wire tip is not satisfactory for the intended selection of the desired branch, the guide wire is evulsed or removed from the catheter, the guide wire tip is reshaped again, and the guide wire is inserted again.

The known types of guide wire include a guide wire in which the superelasticity of a core wire is degraded by a heat treatment or a thermomechanical working. In addition, there is a guide wire in which the surface of a superelastic alloy is plated with a highly malleable metal so that the guide wire can be reshaped.

However, as described in U.S. Pat. Nos. 5,452,726 and 5,069,226, and Japanese Utility Model Publication No. 07-10761, in the case where superelasticity of a core wire is degraded by applying a heat treatment to the core wire, it becomes possible to easily reshape the guide wire tip, but the reshaped shape may be lost upon insertion of the guide wire into a living body. This is because the guide wire tip tends to return to its original straight shape by a shape memory effect. To be more specific, the transformation point of the alloy constituting the core wire is raised by the heat treatment, and the superelasticity of the core wire is not exhibited at room temperature, so that the guide wire tip is reshaped as if it were plastically deformed. However, this is merely an apparent plastic deformation. Therefore, when the guide wire is inserted into the living body and warmed up by the body temperature to approach the transformation point of the alloy, the guide wire tip tends to return to the original straight shape.

In addition, in the case where the superelasticity of a core wire is degraded by a thermomechanical working such as in U.S. Pat. No. 5,238,004, the guide wire tip is not as easy to reshape as expected. Moreover, the worked portion of the core wire is increased in hardness. When a flat plate-like section of a guide wire is made thinner in order to enhance its flexibility, on the other hand, its strength cannot be maintained. The guide wire tip must have a strength (e.g., tensile strength) of not less than a certain value, since it may be moved forward through a stenosis portion while rotating or may be pulled in a bent state. Therefore, there is a limitation to the reduction in thickness of the guide wire tip.

As described in U.S. Pat. Nos. 5,368,049 and 6,234,981, the surface of a superelastic alloy may be plated with a malleable metal, or stainless steel may be vapor deposited on the surface. In these cases, if the surface is coated with the metallic material in such an amount (thickness) as to overcome the superelasticity of the matrix alloy, the distal end part of the guide wire becomes so hard that the flexibility intrinsically required of the guide wire cannot be achieved. On the other hand, if ample flexibility is maintained as a priority, the thickness of the coating material becomes insufficient for overcoming the superelasticity of the matrix alloy, so that the reshapability of the guide wire tip will be unsatisfactory.

### SUMMARY

A guide wire includes a flat plate-shaped section at a distal end section of the guide wire. The surface part on at least one side of the flat plate-shaped section has a residual stress higher than that in an internal or inside part of the flat plate-shaped section. According to one aspect, the surface parts on both sides of the flat plate-shaped section may possess residual stresses higher than the residual stress in the internal part of the flat plate-shaped section.

The distal end section may be fabricated of a NiTi alloy, and the surface part(s) may be imparted with the higher residual stress than the internal part by shot peening. The NiTi alloy constituting the flat plate-shaped section may have a martensite phase. The distal end section of the guide wire may be configured to have a taper section on the proximal side of the flat plate-shaped section, with the taper section having a parent phase of the NiTi alloy. The NiTi alloy forming the distal end section can be selected to have a reverse transformation start temperature lower than room temperature.

The guide wire may further include a wire body, a first resin layer provided on at least a part of the surface of the wire body, and a projecting resin part covering the surface of the first resin layer.

According to another aspect, a guide wire comprises a flat plate-shaped section at a distal end section of the guide wire, wherein the flat plate-like section possesses an internal part and a surface part on both sides of the internal part. The flat plate-shaped section is formed of a NiTi alloy, and the internal part of the flat plate-shaped section has a martensite phase of the NiTi alloy. At least one of the surface parts of the flat plate-shaped section possesses a residual stress higher than the residual stress of the internal part, and the flat plate-shaped section possesses a property allowing the flat plate-shaped section to be shaped by manual finger deformation.

Another aspect involves a method of manufacturing a guide wire. The method may comprise preparing a wire made of a NiTi alloy, pressing a part of the wire to form a flat plate-shaped section, heating the flat plate-shaped section during or after the pressing, and imparting a residual stress to at least a part of a surface part or parts of the flat plate-shaped section.

The imparting of the residual stress can involve imparting a residual stress to both surfaces of the flat plate-shaped section. The imparting of the residual stress is preferably conducted so that an inside or internal part of the flat plate-shaped section has a residual stress lower than the residual stress in the surface part or parts of the flat plate-shaped section. The residual stress is preferably imparted by shot peening. The residual stress can also be imparted by ion plating. The heating of the flat plate-shaped section is preferably conducted to alter the martensite and austenite phase characteristics of the flat plate-shaped section. The flat plate-shaped section can be heated so that the flat plate-shaped section has a reverse transformation start temperature (austenite phase start temperature) higher than room temperature and lower than a human body temperature, and a reverse transformation finish temperature (austenite phase finish temperature) higher than a human body temperature. Alternatively, the flat plate-shaped section can be heated so that the flat plate-shaped section has a reverse transformation start temperature lower than room temperature and a reverse transformation finish temperature higher than a human body temperature.

### BRIEF DESCRIPTION OF THE DRAWING FIGURES

The foregoing and additional features will become more apparent from the following detailed description considered with reference to the accompanying drawing figures in which like features are designated by like reference numerals.

FIG. 1 is a longitudinal side view, partly in cross-section, of an embodiment of a guide wire disclosed herein.

FIG. 2A is an enlarged view of a part of the embodiment of the guide wire shown in FIG. 1 as seen from the direction of the arrow in FIG. 2B, and FIG. 2B is an enlarged plan view of a portion of the guide wire shown in FIG. 1.

FIG. 3 is an enlarged cross-sectional view of a part of the embodiment of the guide wire shown in FIG. 1.

FIG. 4 is a diagram showing an example of the relationship between the depth and the residual stress in an object material.

FIG. 5 is a longitudinal cross-sectional view of an example of the coating layer shown in FIG. 1, in the case where the coating layer is applied to a medical implement.

FIG. 6 is a longitudinal cross-sectional view of another example of the coating layer shown in FIG. 1, in the case where the coating layer is applied to a medical implement.

FIG. 7 is a longitudinal cross-sectional view of an example of the coating layer shown in FIG. 1, in the case where the coating layer is applied to a guide wire.

### DETAILED DESCRIPTION

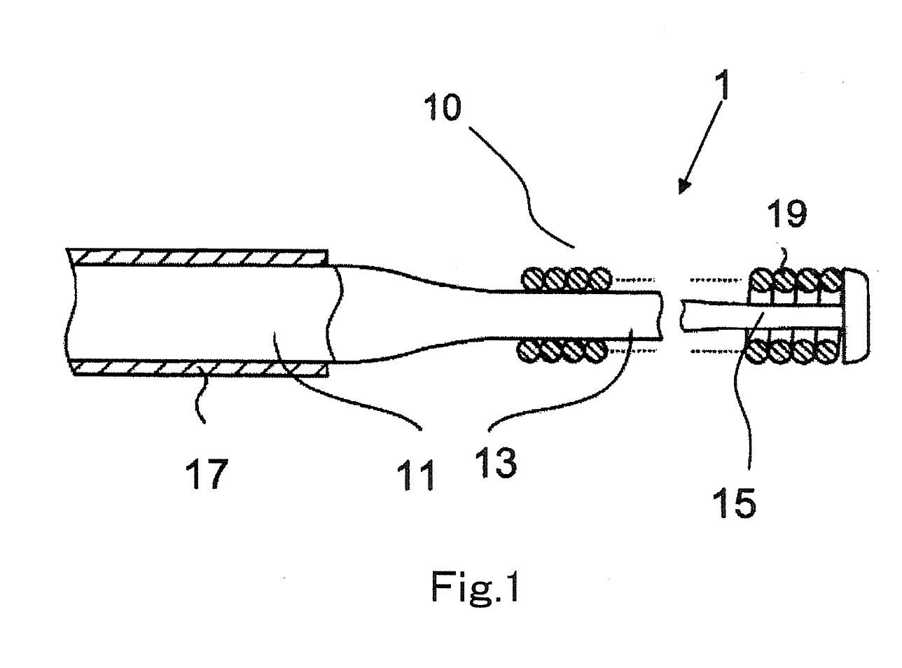

As shown in FIG. 1, the guide wire 1 according to this disclosed embodiment includes a wire body 11, a wire distal end section 13 extending in the distal direction from the distal end of the wire body 11, a flat plate-shaped (i.e., flat plate-like) section 15 positioned at a distal end section 10 of the guide wire 1 and forming a distal end portion of the wire distal end section 13, a coating layer 17 formed on the surface of the wire body 11, and a coil 19 mounted in surrounding or encircling relation to the wire distal end section 13. The wire distal end section 13 positioned on the distal side of the wire body 11 is integrally formed in one piece as a unitary body with the wire body 11.

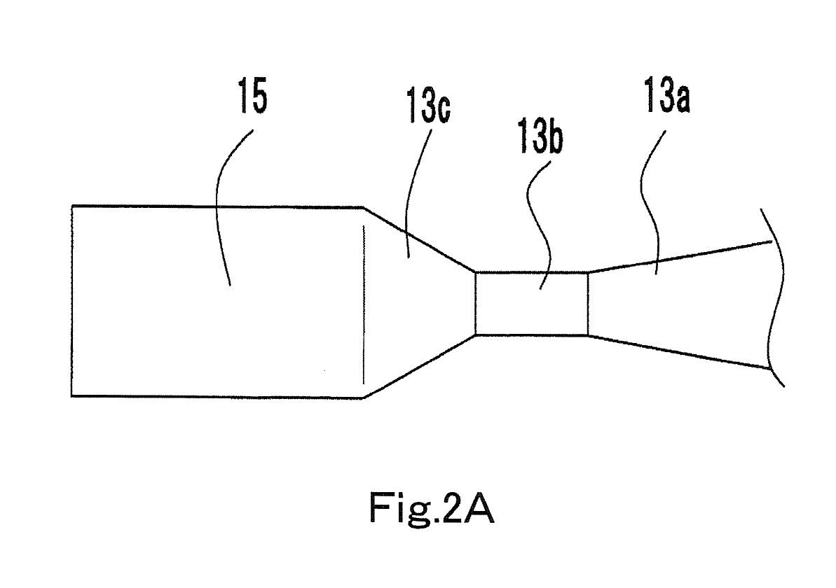

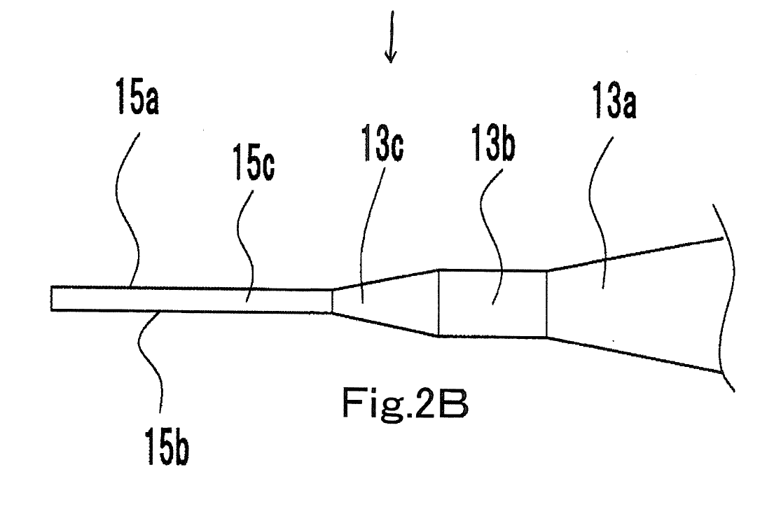

FIG. 2A is an enlarged plan view of a part of the guide wire including the flat plate-shaped section 15 at the distal end section 10 of the guide wire 1 in this embodiment as seen from the direction of the arrow in FIG. 2B. FIG. 2A illustrates the width of the flat plate-shaped section 15 (and the wire distal end section 13), while FIG. 2B illustrates the thickness of the flat plate-shaped section 15 (and the wire distal end section 13). The wire distal end section 13 extends from a taper section 13a to a small diameter section 13b having a substantially uniform outer diameter, and to a wedge section 13c increasing in width while decreasing in thickness along the distal direction. The wedge section 13c is provided on the distal side of the small diameter section 13b, and the flat plate-shaped section 15 is provided on the distal side of the wedge section 13c. The flat plate-shaped section 15 has a surface part 15a, a surface part 15b on the opposite side of the surface part 15a, and an inside part 15c between the one surface part 15a and the other surface part 15b in the thickness direction of the flat plate-shaped section 15.

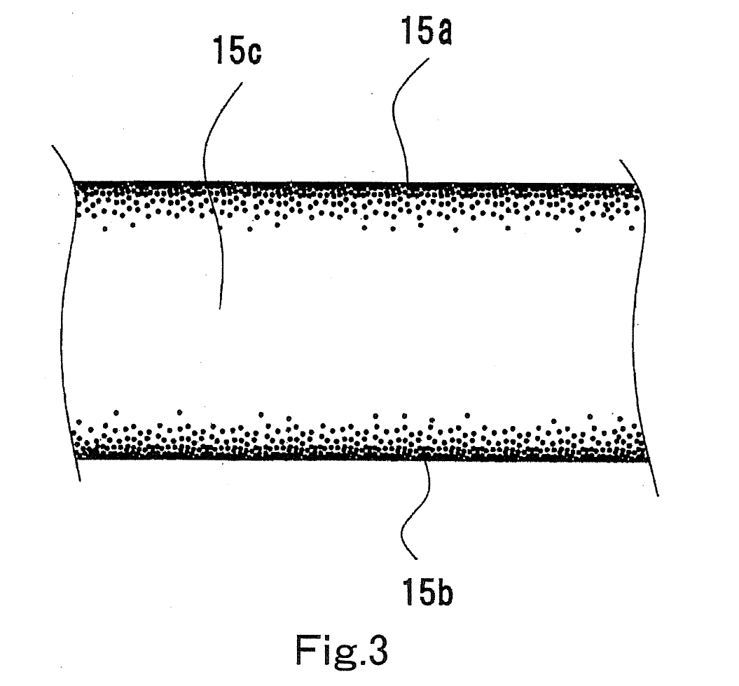

FIG. 3 is an enlarged sectional view of the flat plate-shaped section 15. In FIG. 3, the general distribution of residual stresses is conceptually expressed by use of dots. As shown in FIG. 3, the surface part 15a has a residual stress higher than that in the inside part 15c. The surface part 15b on the other side also has a residual stress higher than that in the inside part 15c. It is preferable that both the one surface part 15a and the other surface part 15b of the flat plate-shaped section 15 have residual stresses higher than the residual stress in the inside part 15c. The thickness of the flat plate-shaped section 15 is 15 to 80 μm, preferably 20 to 60 μm. The depth reached by the residual stress in the surface parts 15a and 15b depends on the thickness of the flat plate-shaped section 15 and the magnitude of the residual stress. In this disclosed embodiment, the depth reached by the residual stress in the surface parts 15a and 15b is 0.5 to 10 μm, preferably 1 to 8 μm. The inside part 15c is higher in hardness than the one surface part 15a and the other surface part 15b.

The one surface part 15a and the other surface part 15b are preferably provided with the residual stress by peening. For example, residual stress is imparted to at least one of the surface part 15a and the surface part 15b, or both, by shot peening.

A configuration may be adopted in which only the one surface part 15a has a residual stress higher than that in the inside part 15c. In this case, residual stress is imparted to only the one surface part 15a by shot peening or the like. In such a situation, the other surface part 15b is on the same level as the inside part 15c with respect to residual stress (i.e., the two have the same residual stress).

It is to be understood that the term “surface part” herein refers to not only the outermost surface but also a part of some depth in the thickness direction, and the term “inside part” refers to the vicinity of the middle point between both the two surface parts 15a, 15b in the thickness direction.

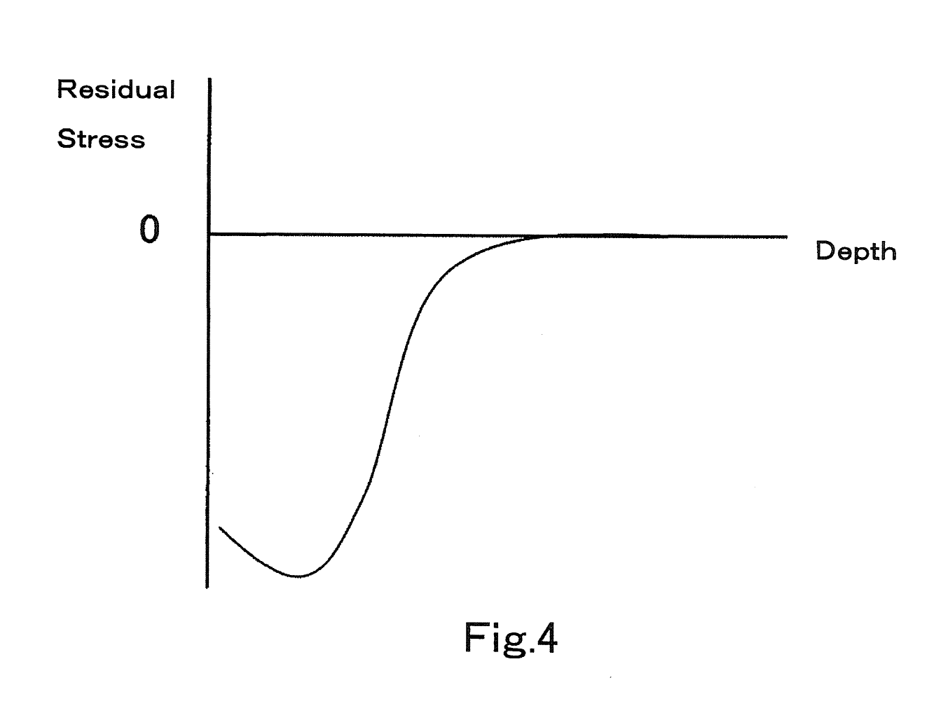

FIG. 4 is a diagram showing an example of the relationship between the depth and the residual stress, in the object material. As mentioned above, examples of the method for imparting the residual stress include shot peening. As shown in FIG. 4, the residual stress is distributed in a concentrated manner in a relatively shallow part from the surface, i.e., in a surface layer part. Many residual stresses are present at the outermost surface part, but the peak of the residual stress is present in the surface layer part below or inside the outermost surface. The residual stress at a given depth decreases steeply as the depth increases beyond the depth where the residual stress peak exists. The residual stresses in the surface part of the guide wire in the above-mentioned embodiment can have the distribution shown in FIG. 4.

The wire distal end section 13 is composed of a NiTi alloy. In the disclosed embodiment shown in FIG. 1, the entire wire body 11 is composed of NiTi alloy. The flat plate-shaped section 15 has a property of being able to be manually reshaped by deformation with fingers. The NiTi alloy constituting the flat plate-shaped section 15 preferably possesses a martensite phase at room temperature, while at least the taper section 13a and the small diameter section 13b of the wire distal end section 13 (and possibly also the wedge section 13c) preferably do not possess a martensite phase at room temperature (i.e., at least the taper section 13a and the small diameter section 13b, and possibly also the wedge section 13c, possess a martensite phase below room temperature). Alternatively, the NiTi alloy constituting the flat plate-shaped section 15 of the wire distal end section 13 may be such that the martensite phase and a parent phase (austenite phase) are at room temperature. As used herein, room temperature means 20° centigrade.

The small diameter section 13b, the taper section 13a and the wedge section 13c of the wire distal end section 13 are preferably of a composition so that the parent (austenite) phase of the NiTi alloy is at room temperature (also at body temperature), whereby the martensite phase (i.e., the austenite phase start and finish temperatures) for the sections 13a, 13b, 13c is below room temperature. According to a further preferred embodiment, the small diameter section 13b and the taper section 13a of the wire distal end section 13 are preferably of a composition so that the parent (austenite) phase of the NiTi alloy is at room temperature (also at body temperature), and the wedge section 13c is preferably of a composition such that both the martensite phase and the parent (austenite) phase of the NiTi alloy is at room temperature so that the austenite start temperature is below room temperature while the austenite finish temperature is above room temperature. The NiTi alloy constituting the flat plate-shaped section 15 preferably has a reverse transformation start temperature (austenite phase start temperature) higher than room temperature and lower than the human body temperature, and a reverse transformation finish temperature (austenite phase finish temperature) higher than the human body temperature. Alternatively, the NiTi alloy constituting the flat plate-shaped section 15 may have a reverse transformation start temperature (austenite phase start temperature) lower than room temperature and a reverse transformation finish temperature (austenite phase finish temperature) higher than the human body temperature. Human body temperature as used herein refers to a temperature of 37° centigrade.

The martensite phase of the NiTi alloy is lower in modulus of elasticity, and hence higher in flexibility, than the parent phase (austenite phase). A NiTi material is more flexible in the martensite phase than in the parent phase (austenite phase). In addition, where the reverse transformation start temperature of the NiTi alloy is higher than room temperature, the alloy is liable to undergo apparent plastic deformation. That is, an NiTi alloy in the martensite phase is deformable and capable of maintaining the deformed shape at temperatures below the reverse transformation temperature. The inside part 15c of the flat plate-shaped section 15 in the martensite phase has more flexibility (enhanced flexibility) than in the parent phase. Though the surface parts 15a and 15b have the martensite phase, manual deformation thereof with fingers is maintained, since these parts contain greater residual stress than the inside part 15c. With the martensite phase of the inside part 15c, the inside part 15c may tend to restore or return to an original rectilinear shape when the guide wire is inserted into a living body and warmed up to the body temperature. However, the internal stress accumulated in the metallic structure of the surface parts 15a, 15b as the residual stress restrains the reverse transformation from the martensite phase to the parent phase (austenite phase), so that the surface parts 15a and 15b are substantially inhibited from exhibiting a shape recovery force. Therefore, a deformation maintaining force of the surface parts 15a and 15b overcomes the shape recovery force of the inside part 15c, whereby the reshaped or deformed shape of the flat plate-shaped section 15 is maintained. Also, in the case where the NiTi alloy constituting the flat plate-shaped section 15 is composed of a combination of the martensite phase and the parent phase at room temperature, the shape recovery force can be weakened to a certain extent. Generally, since the greater the content of the martensite phase the stronger the shape recovery force, the content of the martensite phase is less at room temperature and the shape recovery force is weakened. Therefore, the layers including the residual stress in the surface parts 15a and 15b can be made thinner while securing the reshapability. The residual stress layer(s) may not be necessarily thinner than one including the more content of the martensite phase since the shape recovery force would be weakened. With the guide wire described by way of example above, a physician can change the shape (e.g., deform or bend) the flat plate-shaped section 15 in the desired manner to achieve a shape well suited for facilitating movement within intended portions of the body. In addition, upon being inserted into the body, the flat plate-shaped section 15 is able to maintain the desired deformed/bent shape without returning to the original shape. On the other hand, at least the taper section 13a and the small diameter section 13b (and possibly also the wedge section 13c) exhibit superelasticty after insertion into the body, and preferably prior to insertion into the body. The taper section 13a and the small diameter section 13b (and possibly also the wedge section 13c) maintain the superelasticity after insertion in a body.

The guide wire 1 described above can be manufactured in the following manner. The method generally involves preparing a wire made of a NiTi alloy, pressing a part of the wire to form a flat plate-shaped section, heating the flat plate-shaped section during or after the pressing, and imparting a residual stress to at least a part of a surface part or surface parts of the flat plate-shaped section. The residual stress imparting step is preferably so conducted that the inside part of the flat plate-shaped section becomes softer than the surface part or surface parts. The residual stress imparting step is preferably conducted to impart the residual stress to the surface parts on both sides of the flat plate-shaped section, but may alternatively be conducted to impart the residual stress to only the surface part on one side. In addition, the residual stress imparting step may be performed to impart the residual stress to a central portion or portions in the axial direction of the surface part or surface parts of the flat plate-shaped section. The residual stress imparting step is preferably carried out so that the inside of the flat plate-shaped section has a residual stress lower than the residual stress in the surface part or surface parts. The residual stress imparting step is preferably carried out by peening. Examples of the peening include shot peening. Other methods of imparting the residual stress include at least one selected among ion plating, carburizing, nitriding, sulfurizing and boronizing.

The heating of the flat plate-shaped section 15 is preferably conducted to change the martensite and/or austenite transformation characteristics of at least a part of the flat plate-shaped section to achieve the transformation characteristics mentioned above. According to a preferred embodiment, at least a portion of the remainder of the wire distal section 13 (i.e., at least the taper section 13a and the small diameter section 13b of the wire distal section 13) is not heated. Additionally, it is also possible to not heat the wedge section 13c. The heating of the flat plate-shaped section 15 can include heating the flat plate-shaped section 15 so that the flat plate-shaped section possesses a martensite phase at room temperature. Also, the heating of the flat plate-shaped section 15 can include heating the flat plate-shaped section 15 so that the flat plate-shaped section has a reverse transformation start temperature (austenite phase start temperature) higher than room temperature and lower than a human body temperature, and a reverse transformation finish temperature (austenite phase finish temperature) higher than a human body temperature. Alternatively, the heating of the flat plate-shaped section can involve heating the flat plate-shaped section so that the flat plate-shaped section has a reverse transformation start temperature lower than room temperature and a reverse transformation finish temperature higher than a human body temperature.

The heating of the flat plate-shaped section may be conducted to heat only the surface part on one side of the flat plate-shaped section during or after the pressing so that the surface part on the other side has a residual stress higher than that in the surface part on the one side.

The flat plate-shaped section heating step is preferably conducted so that the reverse transformation start temperature of the flat plate-shaped section is lower than those of the other sections of the guide wire.

The term “flat plate-shaped section” used herein refers to a section that has the capability of being reshaped. The term “flat plate-shaped section” includes a section that is substantially uniform in thickness and width along the distal direction as shown in FIGS. 2A and 2B, and also includes a section which, for example, possesses a thickness and/or width that varies along the length of the section, either in a stepwise manner or a gradual manner.

As shown in FIG. 1, the coating layer 17 of the guide wire 1 may be formed on a part or the entirety of the surface of the wire body 11. The coating layer 17 includes a material possessing excellent hydrophobic and lubricity properties. While the details of the coating layer 17 will be described later, it is noted here that it can be formed from a fluororesin, for example.

The coating layer 17 may be formed not only on the wire body 11 but also on the surface of the coil 19 (and any portion of the wire distal end section 13 not covered by the coil 19). Where the guide wire does not include the coil 19, the coating layer 17 may be formed on the entirety of the surfaces of the wire body 11 and the wire distal end section 13.

In addition, in one preferred embodiment of the guide wire 1, a hydrophilic coating (not shown) may also be formed on a part or the entirety of the surface of the coil 19. The hydrophilic coating may be formed directly on the surface of the coil 19, or may be formed on the coating layer 17 formed on the surface of the coil 19.

The hydrophilic coating is formed from a hydrophilic polymer. Examples of the hydrophilic polymer include polyethylene glycol derivatives, hyaluronic acid, polycarbonates and derivatives thereof, and polyvinyl pyrrolidone and derivatives thereof.

The hydrophilic coating forms a strong water fixation layer on the surface thereof, thereby exhibiting a high affinity for blood in blood vessels and for wall surfaces of the blood vessels and showing a low-friction property (a low coefficient of friction).

The guide wire 1 is preferably tapered along the wire distal end section 13 so that the guide wire can move forward smoothly in a catheter. In this regard, the taper of the guide wire is not limited to a decrease in cross-sectional size of the wire distal end section at a fixed angle as the taper can be configured in other ways to achieve a decrease in cross-sectional size toward the distal end section of the guide wire.

The wire body 11 can be produced by a method in which a first wire including a NiTi alloy and a second wire higher than the NiTi alloy in flexural rigidity are formed separately and thereafter the first wire and the second wire are joined to each other integrally. Examples of the manner of joining the first and second wires integrally include joining the wires by use of a metallic tube, and welding. Examples of welding include laser welding and resistance welding.

The length of the guide wire 1 is not particularly limited. From the viewpoint of steerability or the like, however, the length is preferably 0.3 to 3 meter, more preferably 0.8 to 2 meter.

The outer diameter of the guide wire 1 is also not particularly limited. The outer diameter of the wire body 11 is preferably about 0.2 to 2 mm, and the outer diameter at the distal end of the wire distal end section 13 is preferably about 0.03 to 0.5 mm.

The coil 19 is a coil obtained by forming a wire in a spiral shape, and it is mounted to the wire distal end section 13. The coil 19 imparts appropriate degrees of pliability and rigidity to the distal end section of the guide wire 1.

The cross-sectional shape of the wire constituting the coil 19 is not particularly limited. In order that the guide wire 1 can be moved forward relatively smoothly in a catheter and in blood vessels, the cross-sectional shape is preferably a circle or an ellipse, and is more preferably a circle.

Where the wire forming the coil 19 is circular in cross-section, the diameter of the wire is not particularly limited. From the viewpoint of workability, strength and the like, however, the wire diameter is preferably about 10 to 500 μm.

Where the wire forming the coil 19 is a flat wire (rectangular in cross-section), its thickness is preferably about 10 to 500 μm, and its width is preferably about 20 to 1500 μm.

The length of the coil 19 is not particularly limited. The length in the longitudinal direction of the coil 19 is preferably 10 to 500 mm, and more preferably 30 to 300 mm.

The diameter of the coil 19 is not particularly limited, but it is preferably 0.15 to 3 mm, more preferably 0.2 to 1 mm. The gap between adjacent turns of the coil 19 is also not particularly limited, but it is preferably 0 to 2 mm, more preferably 0 to 0.05 mm.

The material forming the coil 19 is not particularly limited. Examples of the material of the coil 19 include stainless steels, superelastic alloys, cobalt alloys, noble metals such as gold, platinum and tungsten, and alloys thereof. Especially where the coil 19 is formed of a radiopaque material such as noble metals, the guide wire 1 acquires a fluoroscopic contrast property, so that the guide wire 1 can be inserted into a living body while checking the position of the distal end section of the guide wire under fluoroscopy, which naturally is preferable.

In addition, the coil 19 may be formed of different materials on the distal side and on the proximal side thereof. For example, the coil 19 may be composed of a radiopaque material on the distal side and of a material relatively transmissive to X-rays (stainless steel or the like) on the proximal side.

While the outer surface of the wire distal end, inclusive of the flat plate-shaped section, is covered with the coil in the embodiment as above, it may be covered with a plastic jacket. In that case, the outer surface of the wire distal end, inclusive of the flat plate-shaped section, is preferably fixed in intimate contact with a flexible plastic jacket.

The guide wire disclosed here can be used, for example, for treatments as blood vessel dilation and stent indwelling applied to stenosis of a blood vessel such as a coronary artery, treatment of cerebral aneurysm, cerebral thrombosis or the like, a drug injecting treatment applied to portal vein or hepatic arteries in therapy of hepatoma or the like, etc.

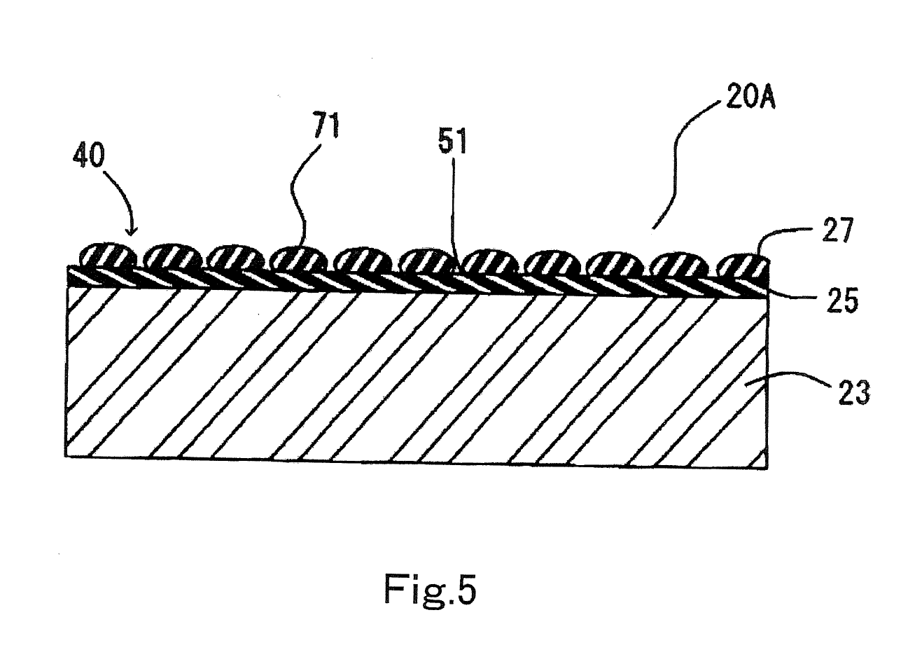

FIG. 5 illustrates in more detail the above-mentioned coating layer 17 shown generally in FIG. 1, as used in connection with a more specific embodiment of a medical implement.

The medical implement 20A in this embodiment is a medical implement 20A provided with a resin coating 40 on the surface thereof. The medical implement includes a base member 23 and a resin coating 40 applied to the base member 23. The resin coating 40 comprises a first resin layer 25 provided on at least a part of the surface of the base member 23, and a projecting resin part (layer) 27 coating or covering the surface of the first resin layer 25. The projecting resin part 27 is configured so that its surface exhibits projected shapes along its longitudinal extent as shown in FIG. 5.

The material forming the base member 23 is not particularly limited, and can be one of various materials. Examples of the material which can be used include metals such as nickel (Ni), titanium (Ti), stainless steels, copper, aluminum, iron, Ni—Ti alloys, cobalt (Co)-based alloys such as Co—Ni-chromium alloys, etc., and resins such as polyimides, polyamides, etc.

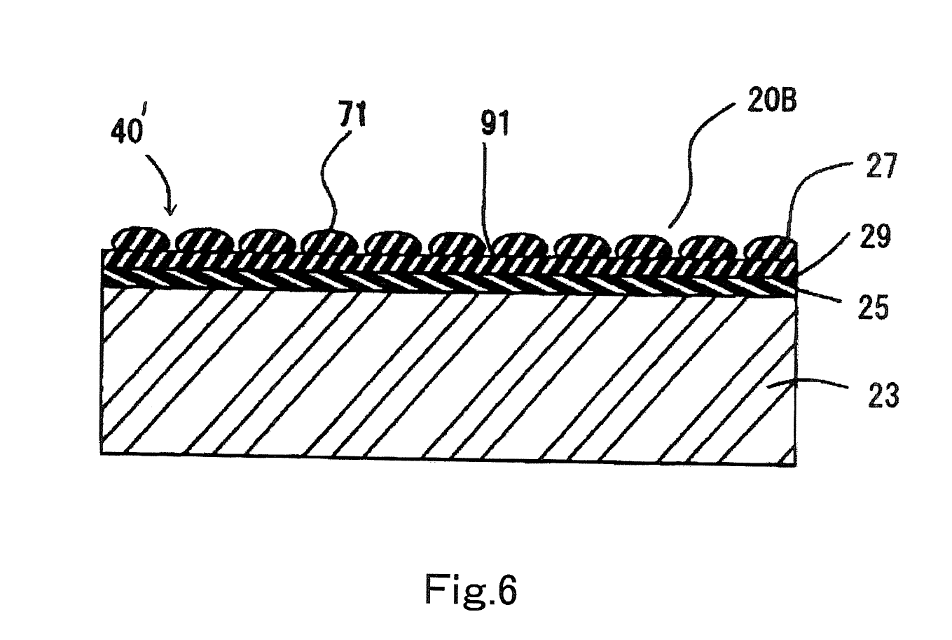

The shape of the base member 23 is also not particularly limited, and can be one of various shapes. Examples of the applicable shapes include wire-like shapes, tubular shapes such as pipes, tubes, etc., flat plate-shaped shapes, thread-like shapes, and other three-dimensional shapes. The base member 23 is preferably a metallic wire, a metallic pipe, a resin tube, or a resin wire. The resin coating 40 (including the embodiment of the coating shown in FIG. 6) can be coated on or applied to the outer surface or the inner surface of the metallic pipe or the resin tube.

The base member 23 in the medical implement 20A is preferably composed of a metal. The base member 23 is preferably a metallic wire or a metallic tubular member. Where the base member 23 is a tubular member, it is preferably a metallic pipe or a metallic coil. The base member 23 is more preferably a wire or pipe made of a nickel-titanium alloy or stainless steel.

Where the medical implement 20A is a catheter, the base member 23 is preferably a metallic pipe, more preferably a nickel-titanium alloy pipe or a stainless steel pipe.

Where the base member 23 is a wire, its diameter is not particularly limited, but the diameter is preferably about 0.1 to 10 mm, more preferably about 0.2 to 1.0 mm.

The first resin layer 25 is provided on at least a part of the surface (outer surface) of the base member 23. Examples of the resin which can be used to form the first resin layer 25 include polyamide-imide resins, epoxy resin, polyphenylene sulfide resin, polyether sulfone resins, polyether ketone resins, polyether amide resins, polysulfone resins, polyimide resins, Parylene resin, and their derivatives. The first resin layer 25 preferably contains the material constituting the projecting resin part 27 which will be described later. Where the material constituting the projecting resin part 27 is a fluororesin, the first resin layer 25 preferably contains a fluororesin.

Though not limited in this regard, the thickness of the first resin layer 25 is 1.0 to 3.0 μm, preferably 1.5 to 2.5 μm.

Various methods can be used to form the first resin layer 25 on at least a part of the surface of the base member 23. For example, the first resin layer 25 can be formed by coating a predetermined region of the surface of the base member 23 with a coating liquid containing the resin for forming the first resin layer 25, followed by drying the applied coating liquid.

On the surface of the first resin layer 25, the projecting resin part 27 having the projected shapes as the surface of the medical implement 20A is provided. Examples of the material which can be used to form the projecting resin part 27 include fluororesins, polyethylene, polyurethane, and polypropylene. Examples of the fluororesins include polytetrafluoroethylene (PTFE), tetrafluoroethylene-perfluoroalkyl vinyl ether copolymer (PFA), polychlorotrifluoroethylene (PCTFE), polyvinylidene fluoride (PVDF), polyvinyl fluoride (PVF), tetrafluoroethylene-hexafluoropropylene copolymer (FEP), and tetrafluoroethylene-ethylene copolymer (PETFE).

The average height of the projections (i.e., the average distance from the outer surface of the first resin layer 25 to the outermost surface of the projections 71) of the projecting resin part 27 is preferably 0.1 to 30 μm. An average height of less than 0.1 μm may lead to an unsatisfactory sliding property, whereas an average height of more than 30 μm may lead to exfoliation of the projecting resin part 27. The average height of the projections refers to the average of the height of several of the projections 71 (e.g., a plurality of randomly selected one of the projections).

The projections 71 of the projecting resin part 27 are present as a multiplicity of projections on a flat or smooth part 51 of the outer surface of the first resin layer 25. The projecting resin part 27 constitutes the outermost surface of the medical implement. In the illustrated embodiment, the projections are spaced apart along the longitudinal extent of the surface of the first resin layer 25. By appropriately selecting the material used to form the projecting resin part 27, a desired surface sliding property can be obtained. The projecting resin part 27 advantageously helps reduce the sliding resistance (friction) relative to objects (e.g., a blood vessel wall or a catheter) which contact the outer surface of the medical implement. At least some of the projections 71 of the projecting resin part 27 may be connected to each other with heating.

The method of forming the projecting resin part 27 is not particularly limited. For example, the projecting resin part 27 can be formed by applying resin particulates to the surface of the first resin layer 25, followed by heating to a temperature not lower than the melting point of the resin. This method helps ensure that a multiplicity of the projections 71 of the projecting resin part 27 project outwardly on the smooth surface of the first resin layer 25.

The heating can be carried out generally by use of a hot gas drying furnace. As an alternative to the hot gas drying furnace, use may be made of high-frequency induction heating or infrared heating. Of the hot gas drying furnaces, indirect heating furnaces are preferred because there is no less concern about entrapment of foreign matter or the like. The high-frequency induction heating is a method of forming a coating film by converting electrical energy into thermal energy from the metal constituting the body being coated. In this method, the resin particles are melted and hardened in sequence from the inside toward the surface of the coating film, so that air and the like present in the coating film can be easily released. This heating method is relatively free of movement of air, is high in energy efficiency, and permits easy control of the heating process. The infrared heating is a method in which infrared energy of, for example, near infrared rays, mid infrared rays or far infrared rays is utilized in the step of melting the resin particles so as to form a uniform coating film.

For example, a coating liquid containing a resin powder having a fixed average particle diameter is prepared, and this coating liquid is applied to the surface of the base member 23. Thereafter, the applied coating liquid is baked by heating to a temperature of not lower than the melting point of the resin powder, whereby projected shapes are formed and fixed to the outermost layer of the coating.

The resin powder is preferably a fluororesin powder in which the resin particulates have a fixed average particle diameter of 3 to 30 μm. If the average particle diameter of the resin particulates is less than 3 μm, the projections in the projecting resin part 27 do not have a desired (sufficient) height sufficient to achieve the desired sliding properties. If the average particle diameter is more than 30 μm, on the other hand, the resin particulates may come off or separate from the first resin layer 25. In the illustrated embodiment shown in FIG. 5, adjacent projections 71 in the projecting resin part are fully spaced from one another (i.e., the adjacent projections 71 are spaced apart from the innermost region of the projecting resin part 27 to the outermost region of the projecting resin part). However, as briefly mentioned above, it is also possible that the plurality of projections 71 in the projecting resin part 27 may be such that at least some adjacent ones of the projections 71 are integrated or connected (cohered) to each other. In such an alternative embodiment, adjacent projections would be integrated with one another in the innermost region of the projecting resin part 27 so that the outermost portions of adjacent projections 71 in the outermost region of the projecting resin part remain spaced apart from one another to continue to provide the reduced sliding resistance characteristics mentioned above.

Incidentally, a third resin layer may be intermediately provided between the base member 23 and the first resin layer 25. If provided, the third resin layer is preferably composed of a thermoplastic resin such as polyurethane and polyethylene. Where the third resin layer is composed of a thermoplastic elastomer, the flexibility of the medical implement 20A can favorably be retained. In the case where the melting point of the resin particulates used in the projecting resin part 27 is higher than the melting point of the third resin layer, the first resin layer shows an adiabatic effect at the time of heating the projecting resin part 27, whereby the third resin layer can be prevented from deteriorating.

FIG. 6 is a cross-sectional view of another example of the medical implement in this embodiment.

The medical implement 20B in this embodiment includes a base member 23 and a coating 40′ applied thereto. The coating 40′ comprises a first resin layer 25 provided on at least a part of the surface of the base member 23, a second resin layer 29 provided on at least a part of the surface of the first resin layer 25, and a projecting resin part 27. The projecting resin part 27 covers the surface of the second resin layer 29 and forms projected shapes along the outer surface of the surface of the medical implement 20B.

The material forming the base member 23, and the configuration or shape of the base member 23 can be similar to those in the above-described embodiment.

The first resin layer 25 is provided on at least a part of the surface (outer surface) of the base member 23. Examples of the resin material which can be used to form the first resin layer 25 include polyamide-imide resins, epoxy resin, polyphenylene sulfide resin, polyether sulfone resins, polyether ketone resins, polyether amide resins, polysulfone resins, polyimide resins, Parylene resin and their derivatives. The first resin layer 25 preferably contains the material constituting the second resin layer 29 which will be described later. Where the material constituting the second resin 29 is a fluororesin, the first resin layer 25 preferably contains a fluororesin.

The method of forming the first resin layer 25 on at least a part of the surface of the base member 23 is not particularly limited. For example, the first resin layer 25 can be formed by coating a predetermined region of the surface of the base member 23 with a coating liquid containing the resin for forming the first resin layer 25, followed by drying the applied coating liquid.

The second resin layer 29 is provided on at least a part of the surface (outer surface) of the first resin layer 25. In FIG. 6, the surface of the first resin layer 25 is entirely covered with the second resin layer 29. However, the surface of the first resin layer 25 may be partly covered with the second resin layer 29. Examples of the resin material which can be used to form the second resin layer 29 include fluororesins, polyethylene, polyurethane, and polypropylene. Fluororesins are particularly preferable as the material for forming the second resin layer 29.

Examples of the fluororesins include polytetrafluoroethylene (PTFE), tetrafluoroethylene-perfluoroalkyl vinyl ether copolymer (PFA), polychlorotrifluoroethylene (PCTFE), polyvinylidene fluoride (PVDF), polyvinyl fluoride (PVF), tetrafluoroethylene-hexafluoropropylene copolymer (FEP), and tetrafluoroethylene-ethylene copolymer (PETFE).

The thickness of the second resin layer 29, though not limited in this regard, is preferably is 1.0 to 3.0 μm, more preferably 1.5 to 2.5 μm.

Various methods can be used to form the second resin layer 29. For example, the second resin layer 29 can be formed by coating a surface (outer surface) of the first resin layer 25 with a dispersion containing the resin for forming the second resin layer 29, and drying the applied dispersion, followed by heating to a temperature of not lower than the melting point of the relevant resin. By heating to a temperature of not lower than the melting point of the relevant resin, a flat or smooth second resin layer 29 can be formed. The heating method for heating the dispersion containing the resin for forming the second resin layer 29, the heating methods mentioned above can be adopted.

The projecting resin part 27 for forming the projected shapes or projections at the surface of the medical implement 20B is provided on the surface (outer surface) of the second resin layer 29. Examples of the resin material which can be used to form the projecting resin part 27 include fluororesins, polyethylene, polyurethane, and polypropylene.

Examples of the fluororesins include polytetrafluoroethylene (PTFE), tetrafluoroethylene-perfluoroalkyl vinyl ether copolymer (PFA), polychlorotrifluoroethylene (PCTFE), polyvinylidene fluoride (PVDF), polyvinyl fluoride (PVF), tetrafluoroethylene-hexafluoropropylene copolymer (FEP), and tetrafluoroethylene-ethylene copolymer (PETFE).

The method for forming the projecting resin part 27 is not particularly limited. For example, the projecting resin part 27 can be formed by applying resin particulates to the surface of the second resin layer 29, followed by heating to a temperature of not lower than the melting point of the relevant resin. The method for heating the resin particulates include those mentioned above.

In a specific example of the forming method, a coating liquid containing a resin powder having a fixed average particle diameter is prepared, and the coating liquid is applied to the surface of the second resin layer 29, followed by drying the applied coating liquid. Thereafter, the dried coating film is baked by heating to a temperature of not lower than the melting point of the resin powder, whereby projected shapes or projections are fixed at the outermost layer of the medical implement 20B.

The average height of projections of the projecting resin part 27 is preferably 0.1 to 30 μm. An average height of less than 0.1 μm may lead to an unsatisfactory sliding property, whereas an average height of more than 30 μm may lead to exfoliation of the projecting resin part 27.

The resin powder is preferably a fluororesin powder in which the resin particulates have a fixed average particle diameter of 3 to 30 μm. If the average particle diameter of the resin particulates is less than 3 μm, the projections in the projecting resin part 27 may not possess the desired height, resulting in a relatively poor sliding property. If the average particle diameter is more than 30 μm, on the other hand, the resin particulates may come off, or separate, from the second resin layer 29. In a manner similar to that described above, the multiplicity of projections 71 in the projecting resin part 27 may be fully spaced apart from one another as illustrated (i.e., from the innermost region of the projecting resin part 27 to the outermost region of the projecting resin part 27), or may be formed so that at least some of the adjacent ones of the projections 71 are integrated or connected to one another at the inner most portion of the resin part 27, while still maintaining a space between adjacent projections in the outermost region of the resin part 27.

In the illustrated embodiment, the projections 71 of the projecting resin part 27 are present as a multiplicity of projections, spaced apart, on a flat or smooth part 91 of the surface of the second resin layer 29.

In addition, a third resin layer may be intermediately provided between the base member 23 and the first resin layer 25. If provided, the third resin layer is preferably composed of a thermoplastic resin such as polyurethane and polyethylene. Where the third resin layer is composed of a thermoplastic elastomer, the flexibility of the medical implement 20B can favorably be retained. In the case where the melting point or flow start temperature of the resin for forming the projecting resin part 27 or the second resin layer 29 is higher than the melting point or flow start temperature of the third resin layer, the first resin layer exhibits an adiabatic effect at the time of heating the projecting resin part 27, whereby the third resin layer can be prevented from deteriorating.

As has been above-mentioned, the medical implement in this embodiment can be used as a medical implement for various purposes by making use of the excellent sliding properties thereof. Specific examples of the applications include artificial organs, stents, catheters, guide wires, orthopedic materials such as implants, and medical implements used in the living bodies such as medical patches, sutures, etc. The medical implement may form a member or a part of a medical device, for example a wire of a stent delivery device. The medical implement in this embodiment is used preferably as a long-size medical implement to be inserted into a body lumen. The medical implement in this embodiment is used preferably as a guide wire or a catheter.

A guide wire, as one of the kinds of medical implements of this embodiment, is described in detail below based on a preferred embodiment shown in the drawings.

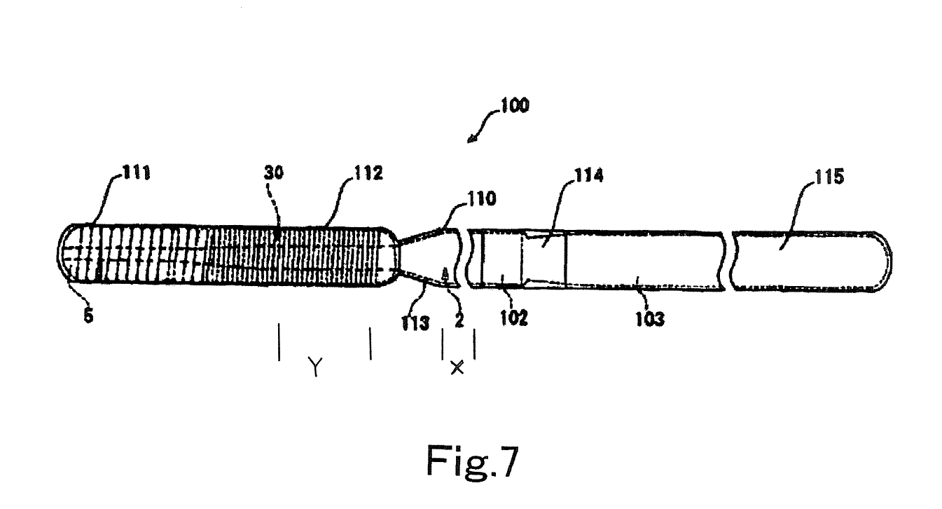

FIG. 7 is a longitudinal sectional view of an example of the guide wire in this embodiment.

The guide wire 100 comprises a core member 2 that includes a distal-side core member 102 and a proximal-side core member 103. The proximal end of the distal-side core member 102 is fixed to the distal end of the proximal-side core member 103. The distal side of the distal-side core member 102 is covered with a coil 30.

The distal-side core member 102 is an elastic wire member. The length of the distal-side core member 102 is not particularly limited, but is preferably about 20 to 1000 mm. In the guide wire 100 in this embodiment, the distal-side core member 102 has a constant outer diameter over a predetermined length from the proximal end of the distal-side core member 102, and its outer diameter gradually decreases along the distal direction from an intermediate portion of the distal-side core member 102.

Various materials can be used for forming the distal-side core member 102. Examples of the material of the distal-side core member 102 include various metallic materials such as stainless steel, among which particularly preferred are pseudo-elastic alloys (inclusive of superelastic alloys), and more preferred are superelastic alloys. Superelastic alloys are comparatively flexible, have a restoring property, and are less liable to acquire a habit of bending. When the distal-side core member 102 is formed from a superelastic alloy, the guide wire 100 can be provided at its distal-side part with sufficient flexibility and a property allowing it to be restored from a bent condition. Therefore, the trackability of the guide wire 100 along blood vessels that are curved or bent in a relatively complicated manner is enhanced, whereby better steerability can be obtained. Further, even when the distal-side core member 102 is repeatedly subjected to curving or bending deformations, the steerability of the guide wire 100 can be prevented from being lowered due to a bending set acquired by the distal-side core member 102 during use of the guide wire 100, since the distal-side core member 102 has a restoring property or ability which inhibits it from acquiring a bending set (i.e., a set bent configuration).

Examples of the preferable composition of the superelastic alloy include NiTi alloys such as a Ni—Ti alloy containing 49 to 52 at. % of Ni, Cu—Zn alloys containing 38.5 to 41.5 wt. % of Zn, and Cu—Zn—X alloys containing 1 to 10 wt. % of X (X is at least one selected from among Be, Si, Sn, Al and Ga), and Ni—Al alloys containing 36 to 38 at. % of Al. Among these, particularly preferred are the Ni—Ti alloys.

The distal end of the proximal-side core member 103 is coupled (connected) to the proximal end of the distal-side core member 102, at a joint section (connecting joint) by welding. The proximal-side core member 103 is an elastic wire member. The length of the proximal-side core member 103 is not particularly limited, but is preferably about 20 to 4800 mm.

The proximal-side core member 103 is formed from a material which is higher than the material constituting the distal-side core member 102 in modulus of elasticity (Young's modulus (modulus of longitudinal elasticity), modulus of rigidity (modulus of transverse elasticity), bulk modulus). This ensures that the proximal-side core member 103 can have an appropriate rigidity (flexural rigidity, torsional rigidity), and the guide wire 100 is relatively firm in bending properties, whereby pushability and torque transmission performance are enhanced, and better insertion steerability can be obtained.

The material (blank material) constituting the proximal-side core member 103 is not particularly limited. Examples of the material which can be used to form the proximal-side core member 103 include various metallic materials such as stainless steel, piano wire, cobalt alloys, and pseudo-elastic alloys.

Where stainless steel is used as the material constituting the proximal-side core member 103, the guide wire 100 possesses better pushability and torque transmission performance.

The distal-side core member 102 and the proximal-side core member 103 are preferably formed from different alloys. In addition, it is preferable that the distal-side core member 102 is formed from a material which is lower in modulus of elasticity than the material of the proximal-side core member 103. This helps ensure that the guide wire 100 possesses properties in which the distal-side part has excellent flexibility while the proximal-side part is rich in rigidity (flexural rigidity, torsional rigidity). As a result, the guide wire 100 possesses excellent pushability and torque transmission performance, whereby good steerability is achieved. At the same time, good flexibility and restoring properties are realized on the distal side, whereby the trackability along blood vessels and safety are enhanced.

In addition, in a specific combination of the distal-side core member 102 with the proximal-side core member 103, it is particularly preferable that the distal-side core member 102 is formed from a Ni—Ti alloy, while the proximal-side core member 103 is formed from a Co—Ni—Cr alloy or stainless steel. This combination provides a guide wire in which the properties mentioned above are highly exhibited.

The guide wire 100 in this embodiment also comprises a coating layer 110 comprised of a plurality of coating parts formed from different coating materials.

The coating layer 110 includes a distal end coating part 111 coating a distal end section of the guide wire inclusive of a distal end of the coil 30 (coil distal end section), a coil coating part 112 coating a part of the coil 30 (i.e., a proximal portion of the coil 30), a distal-side core member proximal end section coating part 113 coating the proximal end section of the distal-side core member 102, a joint section coating part 114 coating the joint section between the distal-side core member 102 and the proximal-side core member 103, and a proximal side core member coating part 115 coating the proximal-side core member 103. The portion of the distal-side core member 102 located inside the coil 30 is not provided with the coating layer 110. That is, in the illustrated embodiment, the coating layer 110 extends distally from the proximal end of the distal-side core member 102 to the point where the coil 30 begins. The sliding property of the distal-side core member proximal end section coating part 113 may be less than the sliding property of the proximal side core member coating part 115, but may be greater than the sliding property of a blank core member or the sliding property of a silicone coated core member.

At least either one of the distal-side core member proximal end section coating part 113 coating the proximal end section of the distal-side core member 102 and the proximal-side core member coating part 115 coating the proximal-side core member 103 is composed of one of the resin coatings 40, 40′ in the embodiments shown in FIGS. 5 and 6 above. Preferably, both the distal-side core member proximal end section coating part 113 and the proximal-side core member coating part 115 are each composed of one of the resin coatings 40, 40′ in the embodiments shown in FIGS. 5 and 6 above.

As an alternative to the embodiment described above, the distal-side core member 102 and the proximal-side core member 103 may be joined to each other by use of a metallic tubular member. In such an alternative embodiment, the resin coating 40, 40′ in the one of the embodiments of FIGS. 5 and 6 above is preferably provided on the surface of the metallic tubular member.

The coil coating part 112 may also be composed of either of the resin coatings 40, 40′ in the embodiment shown in FIGS. 5 and 6 above.

In the case where the distal-side core member 102 is formed from a Ni—Ti alloy, the stress at 3% tensile strain of the distal-side core member 102 at the portion covered with the distal-side core member proximal end section coating part 113 is not less than 85%, preferably not less than 90%, more preferably not less than 95%, based on the stress at 3% tensile strain of the distal-side core member 102 at the portion which is located inside the coil 30 and which is not covered with the coating part 113. In other words, comparing the stress at 3% strain of the distal-side core member 102 in the region X (non-tapered region of the distal-side core member 102) noted in FIG. 7 to the stress at 3% tensile strain of the distal-side core member 102 in the region Y (non-tapered region of the distal-side core member 102), the former is not less than 85%, preferably not less than 90% and more preferably not less than 95%, of the latter. When the just-mentioned stress ratio is not less than 85%, the rigidity of the portion covered with the distal-side core member proximal end section coating part 113 is not significantly deteriorated by the heating during the forming of the coating part 113. The guide wire thus exhibits excellent operational characteristics with respect to pushability and torque transmission performance.

The distal end coating part 111 coats the outer surface of a distal end section of the coil 30 and the outer surface of the portion of a solder 5 fixing the distal end of the distal-side core member 102 and the distal end of the coil 30 to each other.

In addition, at least one of the distal end coating part 111 and the coil coating part 112 is preferably a coating part which exhibits lubricity upon wetting (water absorption). As a material which exhibits lubricity upon wetting (water absorption), many hydrophilic materials can be used. Specific examples of the hydrophilic materials which can be used here include cellulose polymer materials, polyethylene oxide polymer materials, maleic anhydride polymer materials (for example, maleic anhydride copolymer such as methyl vinyl ether-maleic anhydride copolymer), acrylamide polymer materials (for example, polyacrylamide, polyglycidyl methacrylate-dimethylacrylamide (PGMA-DMAA) block copolymer), water-soluble nylons, polyvinyl alcohol, and polyvinyl pyrrolidone.

Additional details associated with this embodiment are described below. A polytetrafluoroethylene resin (PTFE) and a binder resin based on a precursor of a polyamide-imide resin were mixed to prepare an intermediate resin liquid, the viscosity of the intermediate resin liquid was controlled to 30 cP, and a Ni—Ti alloy (Ni: 49-51 at. %) wire with a diameter of 0.340 mm was immersed in the intermediate resin liquid. Then, the wire was drawn out of the intermediate resin liquid, and dried. As a result, a first resin layer with a thickness of 1.5 μm was formed on the wire.

Next, the coated wire was immersed in a PTFE dispersion (31-JR, produced by du Pont) controlled to have a viscosity of 30 cP. Then, the coated wire was drawn out of the dispersion, and baked at 450° C. As a result, a PTFE resin layer with a thickness of 3 μm was formed on the first resin layer.

Subsequently, the wire provided with the PTFE resin layer was immersed in a PTFE powder solution controlled to have a viscosity of 30 cP. Then, the wire was pulled out of the PTFE powder solution, and baked at 450° C. As a result, a projecting resin part of PTFE with a thickness of 5 μm was formed.

The coating solution for forming the projecting resin part was prepared by mixing a PTFE powder (MP1300, a PTFE powder with an average particle diameter of 9 μm, produced by du Pont) with water so as to obtain a solid content of 60 wt. % based on water, and controlling the viscosity of the mixture to 30 cP.

The PTFE resin layer of the PTFE resin coated wire was baked under these conditions, to obtain a final medical wire. Incidentally, a rubbing treatment was not carried out in this example. The coefficient of friction of the medical wire was measured by a frictional feeling tester (KES-SE-SR-U, produced by Kato Tech Co., Ltd.) and was found to be 0.073 on average. Thus, a medical wire with a very low frictional resistance was successfully obtained. In addition, it was found by microscopic observation that a smooth PTFE resin coating film and projected shapes (projections) had been formed at the outermost layer of the medical wire and that the PTFE resin had been baked sufficiently. The Ni—Ti wire showed no influence of heat, and the Ni—Ti wire as the base member retained excellent superelasticity and the uniformity of outer diameter.

The principles, embodiments and modes of operation have been described in the foregoing specification, but the invention which is intended to be protected is not to be construed as limited to the particular embodiments disclosed. The embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A guide wire comprising a flat plate-shaped section at a distal end section of the guide wire, the flat plate-shaped section having opposite sides and an internal part, a surface part on at least one of the sides of the flat plate-shaped section possessing a residual stress higher than the residual stress of the internal part of the flat plate-shaped section.

2. The guide wire as set forth in claim 1, wherein surface parts on both of the sides of the flat plate-shaped section possess residual stresses higher than the residual stress in the internal part of the flat plate-shaped section.

3. The guide wire as set forth in claim 1, wherein the distal end section of the guide wire is comprised of a NiTi alloy.

4. The guide wire as set forth in claim 3, wherein the NiTi alloy constituting the flat plate-shaped section has a martensite phase at 20° centigrade.

5. The guide wire as set forth in claim 4, wherein the NiTi alloy constituting the flat plate-shaped section has a reverse transformation start temperature lower than 20° centigrade.

6. The guide wire as set forth in claim 3, wherein the distal end section has a taper section on the proximal side of the flat plate-shaped section, and the taper section possesses a parent phase of the NiTi alloy at 20° centigrade.

7. The guide wire as set forth in claim 1, wherein the surface part on the one side of the flat plate-shaped section is shot-preened so that it possesses residual stress higher than the residual stress of the internal part of the flat plate-shaped section.

8. The guide wire as set forth in claim 1, further comprising a wire body, a first resin layer provided on at least a part of an outer surface of the wire body, and a projecting resin part covering an outer surface of the first resin layer, the projecting resin part comprising a plurality of spaced apart outwardly extending projections.

9. A guide wire comprising a flat plate-shaped section at a distal end section of the guide wire, the flat plate-shaped section possessing an internal part and a surface part on both sides of the internal part, the flat plate-shaped section being formed of a NiTi alloy, the internal part of the flat plate-shaped section having a martensite phase of the NiTi alloy, at least one of the surface parts of the flat plate-shaped section possessing a residual stress higher than the residual stress of the internal part, the flat plate-shaped section possessing a property allowing the flat plate-shaped section to be shaped by manual finger deformation.

10. The guide wire as set forth in claim 9, wherein the guide wire comprises a wire body possessing a distal end and a wire distal end section extending in a distal direction from the distal end of the wire distal end section, the flat plate-shaped section forming the distal end portion of the wire distal end section, the flat plate-shaped section possessing a reverse transformation start temperature different from the reverse transformation start temperature of another portion of the wire distal end section.

11. A method of manufacturing a guide wire comprising:
preparing a wire made of a NiTi alloy;
pressing a part of the wire made of the NiTi alloy to form a flat plate-shaped section;
heating the flat plate-shaped section during or after the pressing; and
imparting a residual stress to at least a portion of a surface part on at least one side of the flat plate-shaped section.

12. The method of manufacturing a guide wire as set forth in claim 11, wherein the residual stress is imparted to surface parts on both sides of the flat plate-shaped section.

13. The method of manufacturing a guide wire as set forth in claim 11, wherein the residual stress is imparted so that an internal part of the flat plate-shaped section has a residual stress lower than the residual stress in the surface part of the flat plate-shaped section.

14. The method of manufacturing a guide wire as set forth in claim 11, wherein the residual stress is imparted by shot peening.

15. The method of manufacturing a guide wire as set forth in claim 11, wherein the residual stress is imparted by ion plating.

16. The method of manufacturing a guide wire as set forth in claim 11, wherein the heating of the flat plate-shaped section is conducted so that the flat plate-shaped section includes a martensite phase at 20° centigrade.

17. The method of manufacturing a guide wire as set forth in claim 11, wherein the heating of the flat plate-shaped section is conducted so that the flat plate-shaped section has a reverse transformation start temperature lower than 20° centigrade.

18. The method of manufacturing a guide wire as set forth in claim 11, wherein the heating of the flat plate-shaped section is conducted so that the flat plate-shaped section has a reverse transformation start temperature lower than the reverse transformation start temperature of other sections of the wire made of the NiTi alloy.
